(19) United States
(12) Patent Application Publication
(10) Pub. No.: US 2021/0113524 A1
(43) Pub. Date: Apr. 22, 2021

(54) METHODS OF TREATING MYELOPROLIFERATIVE DISORDERS

(71) Applicants: The Henry M. Jackson Foundation for the Advancement of Military Medicine, Inc., Bethesda, MD (US); VIRGINIA COMMONWEALTH UNIVERSITY, Richmond, VA (US)

(72) Inventors: Regina M. DAY, Bethesda, MD (US); Seth J. COREY, Richmond, VA (US)

(21) Appl. No.: 17/058,240

(22) PCT Filed: May 23, 2019
(86) PCT No.: PCT/US2019/033739

§ 371 Date: Nov. 24, 2020

Related U.S. Application Data
(60) Provisional application No. 62/678,376, filed on May 31, 2018.

(51) Int. Cl. A61K 31/401; A61P 35/00

(52) U.S. Cl. CPC A61K 31/401; A61P 35/00

(57) ABSTRACT

The present disclosure provides methods of treating myeloproliferative neoplasm in a subject in need thereof, the method comprising administering to the subject a compound chosen from angiotensin converting enzyme (ACE) inhibitors, angiotensin receptor blockers (ARBs), and renin inhibitors, wherein the compound is administered in an amount effective to treat the myeloproliferative neoplasm in the subject. Also disclosed herein are methods of stabilizing megakaryocytes, at least one hematopoietic growth factor and/or at least one serum amyloid A (SAA) in a patient having a myeloproliferative neoplasm, the method comprising administering to the patient a compound chosen from ACE inhibitors, ARBs, and renin inhibitors, wherein the compound is administered in an amount effective to stabilize megakaryocytes, the at least one hematopoietic growth factor and/or the at least one serum amyloid A (SAA) in the patient.

### CROSS REFERENCE TO RELATED APPLICATIONS

This application claims priority to U.S. Provisional Patent Application No. 62/678,376 filed on 31 May 2018, the entire contents of which are incorporated herein by reference in its entirety.

### GOVERNMENT INTEREST

This invention was made in part with Government support under grant number RO1 HL128173 awarded by the National Institutes of Health. The Government has certain rights in the invention.

The work disclosed herein was funded in part through a grant awarded by the Leukemia & Lymphoma Society.

### SEQUENCE LISTING

The instant application contains a Sequence Listing, which has been submitted via EFS-Web and is hereby incorporated by reference in its entirety. The ASCII copy of the Sequence Listing, created on May 15, 2019, is named HMJ-160-PCT SL.txt, and is 3 kilobytes in size.

### FIELD

This application generally relates to methods of treating myeloproliferative disorders.

### BACKGROUND

Myeloproliferative neoplasms are diseases of the bone marrow characterized by the production of an excess of cells. Primary myelofibrosis, a subset of myeloproliferative neoplasms, is a life-threatening disease with a median survival of 3.5 to 5.5 years [Passamonti, F. et al., Impact of ruxolitinib on the natural history of primary myelofibrosis: a comparison of the DIPSS and the COMFORT-2 cohorts, BLOOD 2014; 123:1833-1835]. Allogeneic stem cell transplantation is currently the only curative therapy for primary myelofibrosis, but because of co-morbidities and limited donor availability, its application is limited [Kroger, N. M. et al., Indication and management of allogenic stem cell transplantation in primary myelofibrosis: a consensus process by an EBMT/ELN international working group, LEUKEMIA 2015; 29:2126-2133].

Gene sequencing of patients with myeloproliferative neoplasms, including primary myelofibrosis, has revealed mutations in the Janus kinase 2 gene (JAK2), the thrombopoietin receptor gene (MPL), and the calreticulin (CALR) gene. To date, however, no pharmaceutical compositions have been approved for curing primary myelofibrosis. The JAK2 inhibitor ruxolitinib is approved only for palliation of symptoms associated with splenomegaly and fatigue, but there is no evidence that JAK2 inhibitors such a ruxolitinib can reverse myelofibrosis [Harrison, C. N. et al., Long-term findings from COMFORT-IL a phase 3 study of ruxolitinib vs best available therapy for myelofibrosis, LEUKEMIA 2016; 30:1701-1707]. Other JAK2 inhibitors have been evaluated in clinical trials but have displayed toxicities [Bose, P. et al., JAK2 inhibitors for myeloproliferative neoplasms: what is next?, BLOOD 2017; 130:115-125]. Moreover, ruxolitinib therapy frequently must be withdrawn due to side effects, such as anemia, thrombocytopenia, and infections.

Thus, novel, non-toxic therapies are needed for the treatment of myeloproliferative neoplasms, including primary myelofibrosis.

### SUMMARY

One aspect of the present disclosure is directed to methods of treating a myeloproliferative neoplasm in a subject in need thereof, the method comprising administering to the subject a compound chosen from angiotensin converting enzyme (ACE) inhibitors, angiotensin receptor blockers (ARBs), and renin inhibitors, wherein the compound is administered in an amount effective to treat the myeloproliferative neoplasm in the subject.

Another aspect of the present disclosure is directed to methods of stabilizing white blood cell numbers and/or stabilizing the levels of Interleukin-9 (IL-9) and Stem Cell Factor (SCF) in a patient having a myeloproliferative neoplasm, the method comprising administering to the patient a compound chosen from ACE inhibitors, ARBs, and renin inhibitors, wherein the compound is administered in an amount effective to stabilize white blood cell numbers and/or stabilize the levels of IL-9 and/or SCF in the patient.

In yet another embodiment of the disclosure, there is provided a method of stabilizing at least one hematopoietic growth factor and/or at least one serum amyloid A (SAA) protein in a patient having a myeloproliferative neoplasm, the method comprising administering to the patient a compound chosen from ACE inhibitors, ARBs, and renin inhibitors, wherein the compound is administered in an amount effective to stabilize the at least one hematopoietic growth factor and/or the at least one SAA protein in the patient. In certain embodiments, the at least one hematopoietic growth factor is selected from the group consisting of EPO and G-CSF. In certain embodiments, the at least one hematopoietic growth factor is G-CSF. In certain embodiments, the at least one SAA protein is SAA1.

In some embodiments, the white blood cells are one or more of eosinophils, neutrophils, or lymphocytes. In certain embodiments, the myeloproliferative neoplasm is chosen from chronic myeloid leukemia, polycythemia vera, essential thrombocytosis, myelofibrosis, chronic neutrophilic leukemia, chronic eosinophilic leukemia, and hypereosinophilic syndrome. In certain embodiments, the myeloproliferative neoplasm is myelofibrosis, and the myelofibrosis is primary myelofibrosis; in certain embodiments, the myeloproliferative neoplasm is myelofibrosis, and the myelofibrosis is secondary myelofibrosis.

In certain embodiments, the compound is an ACE inhibitor, and in certain embodiments, the ACE inhibitor is captopril. In certain embodiments of the methods disclosed herein, the subject is a mammal, and in certain embodiments, the mammal is a human.

In some embodiments of the methods disclosed herein, the administration of the compound stabilizes expression of CD41 and/or CD61 proteins in at least one of bone marrow cells and spleen cells of the subject. In certain embodiments, the administration of the compound stabilizes expression of Col1a and/or Col3a2 in at least one of bone marrow cells and spleen cells of the subject. In certain embodiments, the administration of the compound stabilizes reticulin and/or collagen production in at least one of bone marrow and spleen of the subject.

In certain embodiments, the compound is administered in an amount effective to stabilize splenomegaly in the subject, and in certain embodiments, the compound is administered in an amount effective to stabilize bone marrow fibrosis in the subject.

Another aspect of the present disclosure is directed to a method of stabilizing megakaryocytes in at least one of bone marrow and spleen in a patient having a myeloproliferative neoplasm, the method comprising administering to the patient a compound chosen from ACE inhibitors, ARBs, and renin inhibitors, wherein the compound is administered in an amount effective to stabilize megakaryocytes in at least one of bone marrow and spleen of the patient.

In certain embodiments of the method of stabilizing megakaryocytes in at least one of bone marrow and spleen in a patient having a myeloproliferative neoplasm, the compound is an ACE inhibitor, and in certain embodiments, the compound is captopril. In certain embodiments, the myeloproliferative neoplasm is primary myelofibrosis.

### BRIEF DESCRIPTION OF THE DRAWINGS

The accompanying drawings, which are included to provide a further understanding of the disclosure, are incorporated in and constitute a part of this specification. The drawings illustrate embodiments of the disclosure and, together with the detailed description, serve to explain the principles of the disclosure. No attempt is made to show structural details of the disclosure in more detail than may be necessary for a fundamental understanding of the disclosure and various ways in which it may be practiced.

FIG. 1 is a schematic illustrating the renin-angiotensin-aldosterone (RAAS) system and the effects of ACE inhibitors, ARBs, and renin inhibitors on the RAAS system.

FIG. 2A shows hematoxylin and eosin (H&E) staining at a magnification of 40× of bone marrow from the humeri of one mouse from each of the wild-type, untreated Gata1^{low} mice, and Gata1^{low} mice treated for two months with captopril, as discussed in Example 1.

FIG. 2B shows Gomori staining at a magnification of 60× of the same humeri histological sections shown in FIG. 1A for each of the three mouse cohorts (wild-type, untreated Gata1^{low} mice, and Gata1^{low} mice treated for two months with captopril), as discussed in Example 1.

FIG. 3 is a scatter plot graph showing the bone marrow reticulin scores for wild-type mice, untreated Gata1^{low} mice, and Gata1^{low} mice treated for two months with captopril, as discussed in Example 1.

FIG. 4A shows H&E staining at a magnification of 60× of spleen histological sections of one mouse from each of the wild-type, untreated Gata1^{low} mice, and Gata1^{low} mice treated for two months with captopril, as discussed in Example 1.

FIG. 4B shows Gomori staining at a magnification of 60× of the same spleen histological sections shown in FIG. 3A for each of the three mouse cohorts (wild-type, untreated Gata1^{low} mice, and Gata1^{low} mice treated for two months with captopril), as discussed in Example 1.

FIG. 5 is a scatter plot graph showing the spleen weights for wild-type mice, untreated Gata1^{low} mice, and Gata1^{low} mice treated for two months with captopril, as discussed in Example 1, wherein * indicates a p-value <0.05.

FIG. 6 is a scatter plot graph showing the white blood cell count for wild-type mice, untreated Gata1^{low} mice, and Gata1^{low} mice treated for two months with captopril, as discussed in Example 2, wherein * indicates a p-value <0.05.

FIG. 7 is a scatter plot graph showing the lymphocyte count for wild-type mice, untreated Gata1^{low} mice, and Gata1^{low} mice treated for two months with captopril, as discussed in Example 2, wherein * indicates a p-value <0.05.

FIG. 8 is a scatter plot graph showing the eosinophil count for wild-type mice, untreated Gata1^{low} mice, and Gata1^{low} mice treated for two months with captopril, as discussed in Example 2, wherein * indicates a p-value <0.05.

FIG. 9 is a scatter plot graph showing the neutrophil count for wild-type mice, untreated Gata1^{low} mice, and Gata1^{low} mice treated for two months with captopril, as discussed in Example 2, wherein * indicates a p-value <0.05.

FIG. 10 is a scatter plot graph showing the platelet count for wild-type mice, untreated Gata1^{low} mice, and Gata1^{low} mice treated for two months with captopril, as discussed in Example 2, wherein * indicates a p-value <0.05.

FIG. 11 is a scatter plot graph showing the red blood cell count for wild-type mice, untreated Gata1^{low} mice, and Gata1^{low} mice treated for two months with captopril, as discussed in Example 2.

FIG. 12 is a scatter plot graph showing the percentage of CD45+ cells expressing CD41+ in femur bone marrow for wild-type, Gata1^{low} untreated mice, and Gata1^{low} captopril-treated mice, as discussed in Example 3, wherein * indicates a p-value <0.05.

FIG. 13 is a scatter plot graph showing the fold change in expression of CD41 mRNA in femur bone marrow for wild-type, Gata1^{low} untreated mice, and Gata1^{low} captopril-treated mice, as discussed in Example 3, wherein * indicates a p-value <0.05.

FIG. 14 is a scatter plot graph showing the fold change in expression of CD61 mRNA in femur bone marrow for wild-type, Gata1^{low} untreated mice, and Gata1^{low} captopril-treated mice, as discussed in Example 3, wherein * indicates a p-value <0.05.

FIG. 15 is a scatter plot graph showing the fold change in expression of Col1a mRNA in femur bone marrow for wild-type, Gata1^{low} untreated mice, and Gata1^{low} captopril-treated mice, as discussed in Example 3, wherein * indicates a p-value <0.05.

FIG. 16 is a scatter plot graph showing the fold change in expression of Col3a2 mRNA in femur bone marrow for wild-type, Gata1^{low} untreated mice, and Gata1^{low} captopril-treated mice, as discussed in Example 3, wherein * indicates a p-value <0.05.

FIG. 17 is a scatter plot graph showing the percentage CD45+ cells expressing CD41+ in spleen cells for wild-type, Gata1^{low} untreated mice, and Gata1^{low} captopril-treated mice, as discussed in Example 4, wherein * indicates a p-value <0.05.

FIG. 18 is a scatter plot graph showing the fold change in expression of CD41 mRNA in spleen-derived cells for wild-type, Gata1^{low} untreated mice, and Gata1^{low} captopril-treated mice, as discussed in Example 4, wherein * indicates a p-value <0.05.

FIG. 19 is a scatter plot graph showing the fold change in expression of CD61 mRNA in spleen-derived cells for wild-type, Gata1^{low} untreated mice, and Gata1^{low} captopril-treated mice, as discussed in Example 4, wherein * indicates a p-value <0.05.

FIG. 20 is a scatter plot graph showing the fold change in expression of Col1a mRNA in spleen-derived cells for wild-type, Gata1^{low} untreated mice, and Gata1^{low} captopril-treated mice, as discussed in Example 4, wherein * indicates a p-value <0.05.

FIG. 21 is a scatter plot graph showing the fold change in expression of Col3a2 mRNA in spleen-derived cells for wild-type, Gata1^{low} untreated mice, and Gata1^{low} captopril-treated mice, as discussed in Example 4.

FIG. 22 is a graph showing erythropoietin (EPO) levels post-irradiation for sham mice receiving no radiation, control mice receiving 7.9 Gy total body radiation and a vehicle treatment, and test mice receiving 7.9 Gy total body irradiation and 13 mg/mg/day of captopril administered for 14 days, beginning 48 hours after irradiation, as discussed in Example 5. * indicates p<0.05 between vehicle and sham; † indicates p<0.05 between captopril and sham, and ‡ indicates p<0.05 between vehicle and captopril.

FIG. 23 is a graph showing G-CSF levels post-irradiation for sham mice receiving no radiation, control mice receiving 7.9 Gy total body radiation and a vehicle treatment, and test mice receiving 7.9 Gy total body irradiation and 13 mg/mg/day of captopril administered for 14 days, beginning 48 hours after irradiation, as discussed in Example 5. * indicates p<0.05 between vehicle and sham; indicates p<0.05 between vehicle and captopril and § indicates a single subject in the group.

FIG. 24 is a graph showing SAA1 levels post-irradiation for sham mice receiving no radiation, control mice receiving 7.9 Gy total body radiation and a vehicle treatment, and test mice receiving 7.9 Gy total body irradiation and 13 mg/mg/day of captopril administered for 14 days, beginning 48 hours after irradiation, as discussed in Example 5. * indicates p<0.05 between vehicle and sham; † indicates p<0.05 between captopril and sham, and ‡ indicates p<0.05 between vehicle and captopril. § indicates a single subject in the group.

FIG. 25 is a graph showing interleukin-6 (IL-6) levels post-irradiation for sham mice receiving no radiation, control mice receiving 7.9 Gy total body radiation and a vehicle treatment, and test mice receiving 7.9 Gy total body irradiation and 13 mg/mg/day of captopril administered for 14 days, beginning 48 hours after irradiation, as discussed in Example 5. * indicates p<0.05 between vehicle and sham; † indicates p<0.05 between captopril and sham, and ‡ indicates p<0.05 between vehicle and captopril.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

### Definitions

In order that the present invention may be more readily understood, certain terms are first defined. Additional definitions are set forth throughout the detailed description.

The term “effective amount” refers to a dosage or amount of a compound that is sufficient for treating an indicated disorder, condition, or disease such as ameliorate, palliate, lessen, and/or delay one or more of its symptoms. In reference to cancers or other unwanted cell proliferation, an effective amount comprises an amount sufficient to prevent or delay unwanted cell proliferation, to decrease the cell proliferation rate, to cause a tumor to shrink, and/or to decrease the growth rate of the tumor (such as to suppress tumor growth). In some variations, an effective amount is an amount sufficient to prevent or delay occurrence and/or recurrence. An effective amount can be administered in one or more administrations. In the case of cancer, the effective amount of the compound or composition may: (i) reduce the number of cancer cells; (ii) reduce tumor size; (iii) inhibit, retard, slow to some extent and, in some embodiments, stop cancer cell infiltration into peripheral organs; (iv) inhibit (i.e., slow to some extent and in some embodiments stop) tumor metastasis; (v) inhibit tumor growth; (vi) prevent or delay occurrence and/or recurrence of tumor; and/or (vii) relieve to some extent one or more of the symptoms associated with the cancer.

The term “erythropoietin (EPO)” refers to a glycoprotein cytokine, or cell signaling molecule, secreted primarily by the kidney in response to cellular hypoxia. EPO stimulates the production of red blood cells by the bone marrow.

The term “granulocyte colony-stimulating factor (G-CSF)” refers to a glycoprotein cytokine that is a hematopoietic growth factor. G-CSF is known to stimulate the production of granulocytes, including neutrophils, in the bone marrow, which are subsequently released into the blood.

The term “gene expression” refers to the expression level of a gene in a sample. As is understood in the art, the expression level of a gene can be analyzed by measuring the expression of a nucleic acid (e.g., genomic DNA or mRNA) or a polypeptide that is encoded by the nucleic acid.

The term “Interleukin 9 (IL-9)” refers to a cytokine, or cell signaling molecule, that is an interleukin. The cytokine IL-9 is secreted by CD4+ helper cells and serves to regulate a variety of hematopoietic cells. As IL-9 may serve to stimulate cell proliferation and prevent apoptosis, it is known to play a role in tumors that affect the blood, bone marrow, lymph nodes, and lymphatic system.

The term “megakaryocyte” refers to a large bone marrow cell having a lobated nucleus. Megakaryocytes are known in the art to be responsible for the production of platelets in the bone marrow.

The term “myeloproliferative neoplasm” refers to various blood cancers that occur when a subject produces too many white blood cells, red blood cells, and/or platelets. Exemplary myeloproliferative neoplasms may include chronic myeloid leukemia, polycythemia vera, essential thrombocytosis, myelofibrosis (including primary myelofibrosis and secondary myelofibrosis), chronic neutrophilic leukemia, chronic eosinophilic leukemia, and hypereosinophilic syndrome.

The term “pharmaceutically acceptable carrier” or “pharmaceutically acceptable excipient” means solvents, dispersion media, coatings, antibacterial agents and antifungal agents, isotonic agents, absorption delaying agents, and the like, that are compatible with pharmaceutical administration. The use of such media and agents for pharmaceutically active substances is well-known in the art.

The term “serum amyloid A (SAA) protein” refers to a group of apolipoproteins, including SAA1, SAA2, SAA3, and SAA4, that are produced primarily in the liver in response to inflammatory stimuli. Expression of SAA1 and SAA2 may be regulated by proinflammatory cytokines, including IL-1, IL-6, and TNF-α.

The terms “stabilize” and “stabilizing” mean that there is no increase, for example, no statistically significant increase, or that there is a decrease in a value being measured as compared to a preceding value, such as a value measuring weight, quantity, or severity.

The term “stem cell factor (SCF)” refers to a cytokine that binds to the proto-oncogene c-KIT receptor CD117 and may cause certain types of cells to grow. CD117 may be used to identify hematopoietic progenitors in the bone marrow, and SCF, which is known to have a role in hematopoiesis, may be present as a transmembrane protein and/or a soluble protein.

The terms “treatment” or “treating” and the like refer to any treatment of any disease or condition in a mammal, e.g. a human or a mouse, and includes inhibiting a disease, condition, or symptom of a disease or condition, e.g., arresting its development and/or delating its onset or manifestation in the patient or relieving a disease, condition, or symptom of a disease or condition, e.g., causing regression of the condition or disease and/or its symptoms.

Disclosed herein is are methods of treating a myeloproliferative neoplasm in a subject in need thereof. Also disclosed herein are methods of stabilizing white blood cell numbers and/or reducing the levels of IL-9 and SCF in a patient having a myeloproliferative neoplasm, as well as methods of reducing megakaryocytes in at least one of bone marrow and spleen in a patient having a myeloproliferative neoplasm.

### Myeloproliferative Neoplasms

Myeloproliferative neoplasms are a type of blood cancer resulting in the over-production of white blood cells, red blood cells, and/or platelets in the bone marrow. This over-production of blood cells may result in various symptoms, including bone marrow fibrosis, chronic inflammation, splenomegaly, and/or hepatomegaly. The only curative therapy currently available to patients diagnosed with a myeloproliferative neoplasm is a bone marrow transplant.

Myeloproliferative neoplasms take many forms, and may include, for example, chronic myeloid leukemia, polycythemia vera, essential thrombocytosis, myelofibrosis, chronic neutrophilic leukemia, chronic eosinophilic leukemia, and hypereosinophilic syndrome. In certain embodiments, the myeloproliferative neoplasm is myelofibrosis, and the myelofibrosis is primary myelofibrosis. In certain embodiments, the myeloproliferative neoplasm is myelofibrosis, and the myelofibrosis is secondary myelofibrosis. Primary myelofibrosis is characterized in that it occurs on its own in a subject, while secondary myelofibrosis occurs as a result of another bone marrow disease.

Myelofibrosis may be characterized by abnormal megakaryocytes (platelet precursor cells), aberrant cytokine production, and bone marrow failure with extramedullary hematopoiesis [Terrefi, A. et al., Myeloproliferative neoplasms: molecular pathophysiology, essential clinical understanding, and treatment strategies, J. CLIN. ONCOL. 2011; 29:573-582]. Stem-cell derived myeloproliferation and abnormal cytokine production may lead to the dysregulation of megakaryocytes and fibrotic remodeling of the bone marrow [Nazha, A. et al., Fibrogenesis in primary myelofibrosis: diagnostic, clinical, and therapeutic implications, ONCOLOGIST 2015; 20:1154-1160]. The degree of collagen fibrosis in the bone marrow can be correlated with the severity of primary myelofibrosis [Nazha, A. et al].

Patients with primary myelofibrosis have been found to harbor reduced levels of the transcription factor GATA1 in megakaryocytes [Vannucchi, A. M. et al., Abnormalities of GATA-1 in megakaryocytes from patients with idiopathic myelofibrosis, AM. J. PATHOL. 2005; 167:849-858]. GATA1 is a hematopoietic master transcription factor that is involved in the differentiation of immature blood cells and provides regulation for both erythroid and myeloid lineages. Due to a deletion in the hypersensitive site of its promoter, which drives its transcription in megakaryocytes, GATA1 deficiency results in aberrant megakaryocytopoiesis, which results in hyperproliferative progenitors, defective terminal differentiation, impaired erythropoiesis, and transient anemia [Liew, C. W. et al., Molecular analysis of the interaction between the hematopoietic master transcription factors GATA-1 and PU1, J. BIOL. CHEM. 2006; 281:28296-306; and Garcia, P. et al., c-Myb and GATA-1 alternate dominant roles during megakaryocyte differentiation, J. THROMB. HAEMOST. 2011; 9:1572-81]. Genetically-engineered mouse models based on JAK2, MPL, or CALR mutations are available. In certain embodiments, a Gata1^{low} mouse may also be used to study myelofibrosis because fibrotic remodeling of the bone marrow microenvironment is observed.

An additional common pathway that leads to myelofibrosis is thought to involve aberrant regulation of TGF-β1 and the subsequent deposition of reticulin and collagen fibrosis [Varricchio, L. et al., Pathological interactions between hematopoietic stem cells and their niche revealed by mouse models of primary myelofibrosis, EXPERT REV. HEMATOL. 2009; 2:315-34]. Recent work suggests that malignant and non-malignant cells may cooperate in this inflammatory process and subsequent fibrosis, and that fibrocytes may play a role in this process [Varstovsek, S. et al., Role of neoplastic monocyte-derived fibrocytes in primary myelofibrosis, J. EXP. MED., 2016; 213:1723-40; and Zingariello, M. et al., A novel interaction between megakaryocytes and activated fibrocytes increases TGB-beta bioavailability in the Gata1(low) mouse model of myelofibrosis, AM. J. BLOOD RES. 2015; 5:34-61]. While the identity of the cell types and the inflammatory cytokines directly responsible for myelofibrotic remodeling are not known, their identification might be useful for developing effective, non-transplant therapies for treating myeloproliferative neoplasms, including primary myelofibrosis.

A number of studies have demonstrated the role of angiotensin II in fibrotic remodeling of the lung, heart, kidney, skin, and liver [Nakayama, H. et al., Macromolecular degradation systems and cardiovascular aging, CIRC RES. 2016; 118:1577-1592; Tan, W. S. D. et al., Targeting the renin-angiotensin system as novel therapeutic strategy for pulmonary diseases, CURR. OPIN. PHARMACOL. 2017; 40:9-17; Stawski, L. et al., MMP-12 deficiency attenuates angiotensin II-induced vascular injury, M2 macrophage accumulation, and skin and heart fibrosis, PLoS ONE 2014; 9:e109763; and Pereira R. M. et al., Renin-angiotensin system in the pathogenesis of liver fibrosis, WORLD J. GASTROENTEROL. 2009; 15:2579-2586]. It has been further demonstrated in a number of animal models that ACE inhibitors can block or reverse fibrotic remodeling through the reduction of angiotensin II maturation [Medhora, M. et al., Dose-modifying factor for captopril for mitigation of radiation injury to normal lung, J RADIAT RES. 2012; 53:633-640; Russo, V. et al., ACE inhibition to slow progression of myocardial fibrosis in muscular dystrophies, TRENDS CARDIOVASC MED. 2017; Deas, S. D. et al., Radiation exposure and lung disease in today's nuclear world, CURR OPIN PULM MED. 2017; 23:167-172; Michel, M. C. et al., Angiotensin II type 1 receptor antagonists in animal models of vascular, cardiac, metabolic and renal disease, PHARMACOL THER. 2016; 164:1-81; and Kim, G. et al., Renin-angiotensin system inhibitors and fibrosis in chronic liver disease: a systematic review, HEPATOL INT. 2016; 10:819-828].

As disclosed herein, ACE inhibitors, such as captopril, as well as ARBs and renin inhibitors, may also be able to slow or reverse myeloproliferative neoplasms such as primary myelofibrosis.

### Angiotensin Converting Enzyme (ACE) Inhibitors, Angiotensin Receptor Blockers (ARBs), and Renin Inhibitors

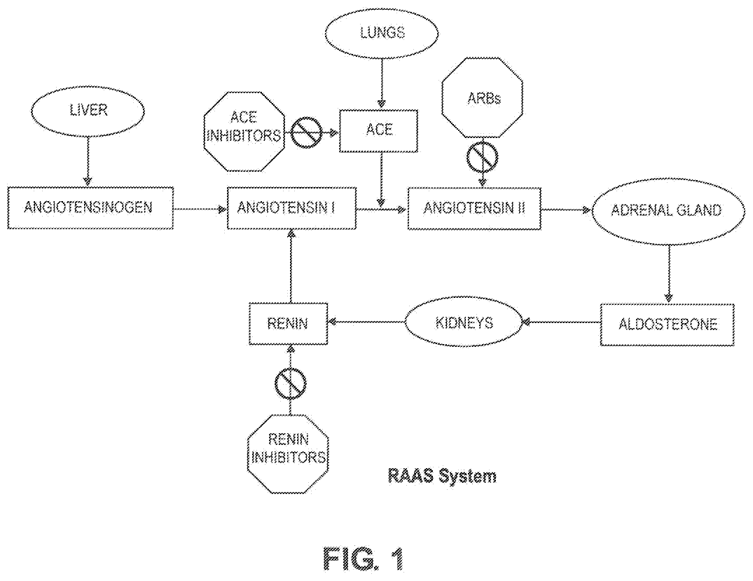

Angiotensin Converting Enzyme (ACE) inhibitors, angiotensin receptor blockers (ARBs), and renin inhibitors all act on the renin-angiotensin-aldosterone (RAAS) system. The RAAS system works to increase low blood pressure and blood volume through vasoconstriction and blood sodium retention. As shown in FIG. 1, the RAAS system begins when the liver produces the enzyme precursor angiotensinogen and the kidney produces renin in response to low fluid volume. Angiotensinogen and renin together produce Angiotensin I. Meanwhile, the lungs release ACE, which together with Angiotensin I produces Angiotensin II. Angiotensin II then acts on the adrenal glands to produce aldosterone, and in turn aldosterone causes vasoconstriction and increases sodium retention in the bloodstream, serving to increase blood pressure and blood volume. Accordingly, ACE inhibitors, ARBs, and renin inhibitors all serve to disrupt the RAAS system at varying points and prevent increases in blood pressure and volume.

In addition to regulating blood pressure and blood volume, components of the RAAS system also regulate the proliferation and maturation of hematopoietic cells [Kim, S. et al., Angiotensin II regulation of proliferation, differentiation, and engraftment of hematopoietic stem cells, HYPERTENSION 2016; 67:574-584]. For example, Angiotensin II modulates the development and proliferation of hematopoietic progenitor cells through Angiotensin II receptors on the cell surface and indirectly regulates EPO. Additionally, ACE is known to regulate other peptides with hematopoietic activities, including, for example, substance P, Ac-SDKP, and angiotensin 1-7 [Shen, X. Z., et al., The peptide network regulated by angiotensin converting enzyme (ACE) in hematopoiesis, CELL CYCLE 2011; 10:1363-69]. Thus, drugs that affect the RAAS system may also have effects, both directly and indirectly, on hematopoietic cell development and proliferation.

Hematopoietic cell development and proliferation is relevant not only to the potential treatment of myeloproliferative neoplasms, but also in the development of countermeasures to treat radiation exposure. The hematopoietic system is uniquely sensitive to radiation damage, including both mature blood cells and hematopoietic stem cells in bone marrow involved in blood cell regeneration. Total body radiation exposure may result in mortality, typically from hematopoietic insufficiency, including severe anemia and leukopenia that may impair immune function, allow life-threatening opportunistic infection, increase vascular permeability, and induce hemorrhage in vital organs. Although the sensitivity of the immune system to radiation is not completely understood, it is believed to be related to the rapid proliferation rates and reduced DNA repair capacity of myeloid/lymphoid hematopoietic progenitors. Thus, discovery of mechanisms of action with respect to treating radiation exposure may also be relevant to the discovery of mechanisms of action with respect to treating myeloproliferative neoplasms, as both are suggestive of the dysregulation of hematopoietic cells.

Angiotensin Converting Enzyme (ACE) inhibitors are pharmaceutical agents that inhibit the angiotensin-converting enzyme, acting to reduce blood volume and dilate blood vessels, which in turn decreases the tension of blood vessels. As such, ACE inhibitors are known for use in the treatment of many conditions, including, for example, hypertension, acute myocardial infarction, cardiac failure such as left ventricular systolic dysfunction, congestive heart failure, renal complication of diabetes mellitus such as diabetic nephropathy, chronic renal failure and renal involvement in systemic sclerosis. In certain instances, ACE inhibitors may be used instead of ARBs and/or renin inhibitors, and in certain embodiments, ACE inhibitors may be used in addition to ARBs and/or renin inhibitors.

It is also known that ACE inhibitors, such as captopril, can reduce the severity of Hematopoietic Syndrome of Acute Radiation Syndrome (H-ARS) in murine models. For example, administration of captopril to mice exposed to total body radiation improved survival rates, in addition to improving blood cell recovery (including of red blood cells, reticulocytes, and platelets), and recovery of colony forming units of granulocyte macrophage (CFU-GM) and megakaryocytes (CFU-M), as well as total colony forming units [Davis, T. A. et al., Timing of captopril administration determines radiation protection or radiation sensitization in a murine model of total body irradiation, EXP. HEMATOL. 2010; 38:270-281].

Although not wishing to be bound by theory, the actions of the ACE inhibitor may be direct, through the reduction of Angiotensin II signaling on hematopoietic progenitors, as well as indirect, through the modulation of cytokine production. As disclosed herein, ACE inhibitor administration may stabilize expression of EPO, SAA, and G-CSF, for example in the treatment of myeloproliferative neoplasms or radiation exposure. See also McCart et al., Delayed captopril administration mitigates hematopoietic injury in a murine model of total body irradiation, SCIENTIFIC REPORTS 2019; 9:2198. It is contemplated that ARB administration and renin inhibitor administration, like ACE inhibitor administration, may likewise stabilize expression of EPO, SAA, and G-CSF, as all are involved in disrupting the RAAS system. See FIG. 1.

EPO and G-CSF are known to stimulate proliferation, survival, and the mobilization of a variety of circulating hematopoietic progenitors [Panopoulos, A. D. et al., Granulocyte colony-stimulating factor: molecular mechanisms of action during steady state and ‘emergency’ hematopoiesis, CYTOKINE 2008; 42:277-288]. In contrast to EPO and G-CSF, which are hematopoietic cytokines, SAA1 is an acute phase protein, primarily produced by the liver, and elevated in the plasma following trauma, infection, inflammatory reactions, and cancer [De Buck, M. et al., Structure and expression of different serum amyloid A (SAA) variants and their concentration-dependent functions during host insults, CURR MED CHEM. 2016; 23:1725-1755; Villapol, S. et al., Hepatic expression of serum amyloid A1 is induced by traumatic brain injury and modulated by telmisartan, AM J PATHOL. 2015; 185:2641-2652]. SAA1 signals through a variety of receptors to regulate downstream pro-inflammatory gene expression [Ye, R. D. et al, Emerging functions of serum amyloid A in inflammation, J LEUKOC BIOL. 98, 923-929 (2015)]. Although not wishing to be bound by theory, it is thought that the suppression of SAA1 may be due to either protection of the liver tissue from radiation damage or suppression of another upstream inflammatory cytokine. Interestingly, SAA1 can induce G-CSF expression [He, R. L. et al., Serum amyloid A induces G-CSF expression and neutrophilia via Toll-like receptor 2, BLOOD 2009; 113:429-437], so reduced SAA may lead to reduced G-CSF.

ACE inhibitors can be divided into three groups based on their molecular structure: (a) sulfhydryl-containing agents including, but not limited to, alacepril, captopril, and zofenopril; (b) dicarboxylate-containing agents including, but not limited to, benazepril, cilazapril, delapril, enalapril, imidapril, lisinopril, moexipril, perindopril, quinapril, ramipril, spirapril, temocapril, trandolapril, and zofenopril; and (c) phosphonate-containing agents including, but not limited to, fosinopril.

In certain embodiments disclosed herein, the ACE inhibitor is captopril. Captopril, otherwise known as 1-[(2S)-3-mercapto-2-methylpropionyl]-L]-proline, is a known suppressor of the renin-angiotensin-aldosterone system that inhibits ACE, a peptidyldipeptide carboxy hydrolase, by preventing the conversion of angiotensin I to angiotensin II.

Angiotensin receptor blockers (ARBs) are also known as angiotensin II receptor antagonists, AT1 receptor antagonists, and sartans. ARBs, like ACE inhibitors, are pharmaceutical agents that modulate the renin-angiotensin-aldosterone system. ARBs block activation of angiotensin II AT1 receptors, which may result in vasodilation, reduced secretion of vasopressin, and reduced production and secretion of aldosterone, among other things. This results in a combined effect of reducing blood pressure. Accordingly, ARBs may be used in the treatment of hypertension, diabetic nephropathy, and congestive heart failure. In certain instances, ARBs may be used instead of ACE inhibitors and/or renin inhibitors, and in certain embodiments, ARBs may be used in addition to ACE inhibitors and/or renin inhibitors.

Examples of ARBs may include, but are not limited to, azilsartan, candesartan, eprosartan, fimasartan, irbesartan, losartan, olmesartan, telmisartan, and valsartan.

Like ACE inhibitors and ARBs, renin inhibitors are pharmaceutical agents that inhibit the renin-angiotensin-aldosterone system by converting angiotensinogen to angiotensin I. Renin inhibitors, like ACE inhibitors and ARBs, may be used to treat hypertension. In certain instances, renin inhibitors may be used instead of ACE inhibitors and/or ARBs, and in certain embodiments, renin inhibitors may be used in addition to ACE inhibitors and/or ARBs. Examples of renin inhibitors may include, for example, aliskiren.

### Methods of Treatment

Disclosed herein are methods of treating a myeloproliferative neoplasm in a subject in need thereof, the method comprising administering to the subject a compound chosen from ACE inhibitors, ARBs, and renin inhibitors, wherein the compound is administered in an amount effective to treat the myeloproliferative neoplasm in the subject. In certain embodiments, the compound is an ACE inhibitor, and in certain embodiments, the ACE inhibitor is captopril.

In certain embodiments, the effect of the ACE inhibitors, ARBs, and/or renin inhibitors on the patient may be measured in the bone marrow or the blood, and in certain embodiments, the effect of the ACE inhibitors, ARBs, and/or renin inhibitors on the patient may be measured in an organ such as the spleen. In certain embodiments, the myeloproliferative neoplasm is chosen from chronic myeloid leukemia, polycythemia vera, essential thrombocytosis, myelofibrosis, chronic neutrophilic leukemia, chronic eosinophilic leukemia, and hypereosinophilic syndrome. In certain embodiments, the myelofibrosis is chosen from primary and secondary myelofibrosis. In certain embodiments of the methods disclosed herein, the compound is an ACE inhibitor, and in certain embodiments, the ACE inhibitor is captopril.

Also disclosed herein are methods of stabilizing white blood cell numbers in a patient having a myeloproliferative neoplasm, the method comprising administering to the patient a compound chosen from ACE inhibitors, ARBs, and renin inhibitors, wherein the compound is administered in an amount effective to stabilize white blood cell numbers. As used herein, stabilizing may indicate a decrease or no substantial further increase in a value. Accordingly, stabilizing white blood cell numbers may, in certain embodiments, indicate decreasing white blood cell numbers, and, in certain embodiments, stabilizing white blood cell numbers may indicate that white blood cell numbers do not further increase compared to a threshold value in a patient. White blood cells may include, for example, neutrophils, eosinophils, basophils, monocytes, and lymphocytes, including T cells and B cells. Thus, in certain embodiments, disclosed herein is a method of stabilizing neutrophils in a patient having a myeloproliferative neoplasm, the method comprising administering to the patient a compound chosen from ACE inhibitors, ARBs, and renin inhibitors, wherein the compound is administered in an amount effective to stabilize neutrophils. In certain embodiments, disclosed herein is a method of stabilizing eosinophils in a patient having a myeloproliferative neoplasm, the method comprising administering to the patient a compound chosen from ACE inhibitors, ARBs, and renin inhibitors, wherein the compound is administered in an amount effective to stabilize eosinophils. In certain embodiments, disclosed herein is a method of stabilizing lymphocytes in a patient having a myeloproliferative neoplasm, the method comprising administering to the patient a compound chosen from ACE inhibitors, ARBs, and renin inhibitors, wherein the compound is administered in an amount effective to stabilize lymphocytes.

In certain embodiments, administering a compound chosen from ACE inhibitors, ARBs, and renin inhibitors to a patient having a myeloproliferative neoplasm stabilizes the levels of IL-9 in the patient. In certain embodiments, administering a compound chosen from ACE inhibitors, ARBs, and renin inhibitors to a patient having a myeloproliferative neoplasm stabilizes the levels of SCF in the patient. In certain embodiments, administering a compound chosen from ACE inhibitors, ARBs, and renin inhibitors to a patient having a myeloproliferative neoplasm stabilizes the levels of both IL-9 and SCF in the patient.

Also disclosed herein are methods of stabilizing at least one hematopoietic growth factor and/or at least one SAA protein in a patient having a myeloproliferative neoplasm, the method comprising administering to the patient a compound chosen from ACE inhibitors, ARBs, and renin inhibitors, wherein the compound is administered in an amount effective to stabilize the levels of the at least one hematopoietic growth factor or the at least one SAA protein. In certain embodiments, the at least one hematopoietic growth factor is selected from the group consisting of EPO and G-CSF. In certain embodiments, the at least one hematopoietic growth factor is G-CSF. In certain embodiments, the at least one SAA protein is SAA1.

In certain embodiments of the methods disclosed herein, the administration of a compound chosen from ACE inhibitors, ARBs, and renin inhibitors to a patient having a myeloproliferative neoplasm stabilizes the reticulin deposition in the patient, such as the reticulin deposition in the bone marrow of the patient or the reticulin deposition in the spleen of the patient. In certain embodiments, the administration of a compound chosen from ACE inhibitors, ARBs, and renin inhibitors to a patient having a myeloproliferative neoplasm stabilizes the collagen production in the patient, such as the collagen production in the bone marrow of the patient or the collagen production in the spleen of the patient.

In certain embodiments, the administration of a compound chosen from ACE inhibitors, ARBs, and renin inhibitors to a patient having a myeloproliferative neoplasm stabilizes the reticulin score of the patient. A reticulin score may be calculated by any means known in the art, including, for example, the method set forth in Kvasnicka, H. M., Problems and pitfalls in grading of bone marrow fibrosis, collagen deposition and osteosclerosis—a consensus-based study, HISTOPATHOLOGY 2016; 68: 905-15. The reticulin score may, for example, range from 0-3, wherein a reticulin score of 0 may indicate normal bone marrow, having scattered linear reticulum with no intersections or the presence of only perivascular collagen, and a reticulin score of 3 may indicate diffuse and dense reticulin with extensive intersections and coarse bundles of collagen.

In certain embodiments of the methods disclosed herein, the administration of a compound chosen from ACE inhibitors, ARBs, and renin inhibitors to a patient having a myeloproliferative neoplasm stabilizes the number megakaryocytes in a patient. In certain embodiments, the megakaryocytes are present in the bone marrow of the patient, and in the certain embodiments, the megakaryocytes are present in the spleen of the patient.

In certain embodiments of the methods disclosed herein, the administration of a compound chosen from ACE inhibitors, ARBs, and renin inhibitors to a patient having a myeloproliferative neoplasm, stabilizes the number of CD41+ megakaryocytes in the patient or stabilizes the expression of CD41 in cells of the patient. In certain embodiments, the administration of a compound chosen from ACE inhibitors, ARBs, and renin inhibitors to a patient having a myeloproliferative neoplasm stabilizes the number of CD61+ megakaryocytes in the patient or stabilizes the expression of CD61 in cells of the patient. In certain embodiments, the cells of the patient are selected from blood cells, bone marrow cells, and spleen cells.

In certain embodiments of the methods disclosed herein, the administration of a compound chosen from ACE inhibitors, ARBs, and renin inhibitors to a patient having a myeloproliferative neoplasm stabilizes the expression of Col1a (including Col1a1 and Col1a2) in cells of the patient. In certain embodiments, the administration of a compound chosen from ACE inhibitors, ARBs, and renin inhibitors to a patient having a myeloproliferative neoplasm stabilizes the expression of Col3a2 in cells of the patient. Col1a2 and Col3a2 are both genes encoding collagen. In certain embodiments, the cells of the patient are selected from blood cells, bone marrow cells, and spleen cells.

Typically, gene expression, such as the expression of CD41, CD61, Col1a2, and Col3a2, can be detected or measured on the basis of mRNA, cDNA, or protein levels. Any quantitative or qualitative method for measuring mRNA levels, cDNA, or protein levels can be used. Suitable methods of detecting or measuring mRNA or cDNA levels include, for example, Northern Blotting, microarray analysis, RNA-sequencing, or a nucleic acid amplification procedure, such as reverse-transcription PCR (RT-PCR) or real-time RT-PCR, also known as quantitative RT-PCR (qRT-PCR). Such methods are well known in the art. See e.g., Sambrook et al., Molecular Cloning: A Laboratory Manual, 4^{th} Ed., Cold Spring Harbor Press, Cold Spring Harbor, N.Y., 2012. Other techniques include digital, multiplexed analysis of gene expression, such as the nCounter® (NanoString Technologies, Seattle, Wash.) gene expression assays, which are further described in US20100112710 and US20100047924.

In certain embodiments, the administration of a compound chosen from ACE inhibitors, ARBs, and renin inhibitors to a patient having a myeloproliferative neoplasm stabilizes splenomegaly in the patient. Splenomegaly, a symptom that may be indicative of certain conditions including myeloproliferative neoplasm, is an abnormal enlargement of the spleen. Splenomegaly may be determined, for example, by palpitation and/or by diagnostic imaging, such as ultrasound scan, computerized tomography (CT) scan, or magnetic resonance imaging (MRI). Accordingly, in certain embodiments, the administration of the compound stabilizes splenomegaly in that the weight and/or size of the spleen decreases or is not further significantly increased.

In certain embodiments, the administration of a compound chosen from ACE inhibitors, ARBs, and renin inhibitors to a patient having a myeloproliferative neoplasm stabilizes bone marrow fibrosis in the patient. Bone marrow fibrosis, or scar tissue formation in the bone marrow, may be characterized by an increase in the deposition of reticulin and collagen fibrosis in the bone marrow. Bone marrow fibrosis may lead to anemia, weakness, fatigue, and splenomegaly. Bone marrow fibrosis may be detected by any means known in the art, such as, for example, bone marrow biopsy and bone marrow aspiration.

### Dosages and Administration

The compounds according to the disclosure may be present in a composition, such as a pharmaceutical composition, useful for treating myeloproliferative neoplasm. In certain embodiments, disclosed herein is a composition comprising an ACE inhibitor such as captopril for use in treating a myeloproliferative neoplasm. In certain embodiments, the compositions are suitable for pharmaceutical use and administration to patients. In addition to a compound chosen from ACE inhibitors, ARBS, and renin inhibitors, the pharmaceutical compositions disclosed herein may further comprise a pharmaceutically acceptable carrier. The pharmaceutical compositions may also comprise other active compounds providing supplemental, additional, or enhanced therapeutic functions. In certain embodiments, the pharmaceutical compositions may also be included in a container, pack, or dispenser, together with instructions for administration.

Pharmaceutically acceptable carriers may include any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in Remington's Pharmaceutical Sciences by E. W. Martin, 18th Edition.

A pharmaceutical composition as disclosed herein is formulated to be compatible with its intended route of administration. Methods to accomplish the administration are known to those of ordinary skill in the art. This includes, for example, administration chosen from intravenously, intravascularly, subcutaneously, intramuscularly, intraperitoneally, intraventricularly, intraepidurally, orally, nasally, ophthalmically, rectally, and topically. Sustained release administration may also be contemplated.

The dosage form of the pharmaceutical composition may comprise conventional oral dosage forms, rectal forms, or parenteral forms. For example, in certain embodiments, the dosage form may be chosen from tablets, capsules, suppositories, powders, ampoules, suspensions, solutions, syrups, sustained release preparations, and liquid injectable forms such as sterile solutions. In certain embodiments, administration is oral, and in certain embodiments, the dosage form is a tablet or a capsule.

The appropriate dosage of the pharmaceutical compositions disclosed herein will depend on various factors, including the type of ACE inhibitor, ARB, or renin inhibitor (or combinations thereof) used, route of administration, frequency of administration, patient's health, age, or size, the type and severity of the myeloproliferative neoplasm to be treated, whether the agent is administered for preventative or therapeutic purposes, previous therapy, the patient's clinical history and response to ACE inhibitors, ARBs, or renin inhibitors, and the discretion of the attending physician.

In certain embodiments, the pharmaceutical composition may be administered daily (e.g., once, twice, thrice, four times, etc. daily), every other day (e.g., once, twice, thrice, four times, etc. every other day), semi-weekly, weekly, once every two weeks, once a month, etc. In certain embodiments, the pharmaceutical composition is administered at least once a day, and in certain embodiments, the pharmaceutical composition is administered at least twice a day. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration is required. Dosage and frequency may vary depending on the patient.

In certain embodiments, the effective amount may fall within the range of about 0.001 mg/kg to about 500 mg/kg, such as from about 0.01 mg/kg to about 50 mg/kg, about 1 mg/kg to about 10 mg/kg, or about 0.01 mg/kg to about 1 mg/kg. In certain embodiments wherein the compound is an ACE inhibitor, the effective amount may fall within the range of about 0.001 mg/kg to about 100 mg/kg, such as from about 0.01 mg/kg to about 50 mg/kg, about 1 mg/kg to about 10 mg/kg, or about 0.01 mg/kg to about 1 mg/kg. In certain embodiments wherein the compound is an ARB, the effective amount may fall within the range of about 0.001 mg/kg to about 100 mg/kg, such as from about 0.01 mg/kg to about 50 mg/kg, about 0.1 mg/kg to about 10 mg/kg, or about 0.01 mg/kg to about 1 mg/kg. In certain embodiments wherein the compound is a renin inhibitor, the effective amount may fall within the range of about 0.001 mg/kg to about 100 mg/kg, such as from about 0.01 mg/kg to about 50 mg/kg or about 1 mg/kg to about 30 mg/kg.

In certain embodiments, an oral dosage form may comprise the ACE inhibitor, ARB, or renin inhibitor in an amount ranging from about 1 mg to about 750 mg, such as from about 125 mg to about 500 mg, from about 25 mg to about 150 mg, or from about 1 mg to about 50 mg. All dosages and regimens are subject to optimization. Optimal dosages can be determined by performing in vitro and in vivo pilot efficacy experiments as is within the skill of the art but taking the present disclosure into account.

In certain embodiments, the methods of treatment disclosed herein may further comprises administering at least one additional active agent, such as at least one additional chemotherapeutic agent. Administration of at least one additional active agent may be simultaneous or sequential to administration of the ACE inhibitor, ARB, or renin inhibitor. In certain embodiments, the at least one additional active agent may be chosen from, for example, JAK2 inhibitors such as arsenic trioxide, azacytidine, cyclophosphamide, cytarabine, dasatinib, daunorubicin, decitabine, doxorubicin, imatinib mesylate, nilotinib, and ruxolitinib.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

Unless indicated otherwise in these Examples, the methods involving commercial kits were done following the instructions of the manufacturers. The following materials and methods refer to Examples 1-4. The materials and methods for Example 5 immediately precede that Example.

Chemicals

Reagents were obtained from Sigma-Aldrich (St. Louis, Mo.) except where indicated.

Animals and ACE Inhibitor Treatment

All animal handling procedures were performed in compliance with guidelines from the National Research Council for the ethical handling of laboratory animals and were approved by the Uniformed Services University of the Health Sciences Institutional Animal Care and Use Committee. Male and female Gata1^{low} and wild type CD1 mice were purchased from Jackson Laboratories (Bar Harbor, Me.). Quantitative PCR confirmed low expression of Gata1. The mice were crossed to a CD1 background to establish a line of homozygous mutant mice. Mice were kept in a barrier facility for animals accredited by the Association for Assessment and Accreditation of Laboratory Animal Care International. Mice were housed in groups of four. Animal rooms were maintained at 21±2° C., 50%±10% humidity, and 12-hour light/dark cycle with commercial, freely-available rodent ration (Harlan Teklad Rodent Diet 8604, Frederick, Md., USA).

Captopril (USP grade; Sigma-Aldrich, St Louis, Mo., USA) was dissolved in acidified water at 0.6 g/L, and provided to animals starting at 10 months of age until 12 months of age, as described in Davis et al., Timing of captopril administration determines radiation protection or radiation sensitization in a murine model of total body irradiation, EXP. HEMATOL. 2010; 38: 270-81. The stability of captopril in acidified water was previously established, as described in Escribano, G. M. J. et al., Stability of an aqueous formulation of captopril at 1 mg/ml, FARM HOSP. 2005; 29: 30-6. Based on previously measured volumes of water consumed per day by the mice, the daily water consumption was determined to be a dose of 79 mg/kg/day [Davis et al., 2010]. Control animals received acidified water (vehicle) without captopril. Animals were euthanized at 13 months of age.

Blood Cell Analysis

Complete blood counts (CBC) with differentials were obtained using a Baker Advia 2120 Hematology Analyzer (Siemens, Tarrytown, N.Y., USA). Separate mice were used for each point (n=5-6 per group).

Histology and Myelofibrosis Scoring

Sternebrae, humeri, and femurs were surgically removed from euthanized animals and fixed in 10% neutral formalin overnight. Tissues were paraffin blocked and stained using standard methods for hematoxylin and eosin (H&E), Masson's trichrome, and Gomori's reticulin stain by Histoserve (Germantown, Md.). Stained slides were evaluated by a pathologist who was blinded to the identity of the treatment groups and using a published system for scoring myelofibrosis [Kvasnicka, H. M., Problems and pitfalls in grading of bone marrow fibrosis, collagen deposition and osteosclerosis—a consensus-based study, HISTOPATHOLOGY 2016; 68: 905-15]. Bone marrow sections were digitally scanned using the Zeiss Axioscan, and images were produced with Zen Lite software (Carl Zeiss, USA).

Bone Marrow and Spleen Isolation

Mice were euthanized with pentobarbital (10 mg/kg). Humeri and femurs were surgically removed from euthanized animals and flushed with sterile phosphate buffered saline (PBS). Spleens were smashed through a 40 μM cell strainer (Cell Treat, Pepperell, Mass.) using the plunger end of a small syringe. The cell strainer was rinsed with PBS (end volume of 30 mL) and cells were collected by centrifugation at 300×g for 10 min at room temperature. Red blood cells were lysed by resuspending bone marrow cells in 2 mL (1 min incubation) or spleen cells in 5 mL of ammonium-chloride-potassium lysis buffer (5 minute incubation). Cells were then diluted in 20 mL PBS, washed twice, and pelleted as before.

Cell Staining and Analysis

Cells isolated from spleen and bone marrow were resuspended in about 200 μl PBS and placed on 5 ml nylon cell strainer topped Falcon tubes (Corning Life Sciences, Corning, N.Y.) and centrifuged for 10 min at 860×g at room temperature. Cells were resuspended in 100 μl PBS and transferred to Falcon 96-well clear V-bottom untreated polypropylene storage microplates (Corning Life Sciences). Cells were then stained with LIVE/DEAD viability stain (Molecular Probes, Life Technology, Grand Island, N.Y.) for 20 minutes in the dark, washed with staining buffer (0.5% FBS, 0.05% NaN₃ in PBS), and pelleted by centrifugation for 5 min at 860×g at room temperature and subsequently blocked by 1 μl Fc Block (BD Bioscience, San Jose, Calif.) diluted in 99 μl staining buffer for 20 minutes on ice. Plates were centrifuged at 860×g for 5 minutes at room temperature, and supernatants were removed. After washing with 200 μl of staining buffer, the cells were stained with a cocktail containing: Brilliant Violet 605-labeled CD45 (1:160, Cat #: 103140, Biolegend, San Diego, Calif.); allophycocyanin (APC)-eFluor 780-labeled CD115 (1:80, Ref #: 47-1152-82, Affymetrix eBioscience, San Diego, Calif.); and R-Phycoerythrin (PE)-labeled CD41 (1:160, Cat #558040, BD Bioscience, San Jose, Calif.) for 20 minutes on ice. After washing, cells were stained with anti-biotin-FITC (1:45, Miltenyi Biotech, San Diego) for 20 minutes on ice. The cells were washed, pelleted, resuspended in Perm/Wash buffer, and analyzed using a BD LSR II flow cytometer (BD Bioscience). Data analysis was carried out with FlowJo data analysis software version 10.1r5 (FlowJo, Ashland, Oregon).

Reverse Transcription Polymerase Chain Reaction (RT-PCR)

Total RNA was extracted from cells isolated from bone marrow or spleen cells using phenol-chloroform extraction with silicone lubricant using a modified protocol as described in Mukhopadhyay, T., Silicone lubricant enhances recovery of nucleic acids after phenol-chloroform extraction, NUCLEIC ACIDS RES. 1993; 21: 781-2.

Approximately 25 mg of tissue was homogenized in 1 ml of Trizol reagent. After the addition of 200 μl of chloroform, 125 μl of RNAse free water was added. Samples were added to prepared tubes, and centrifuged at 12,000 rpm for 15 minutes at 4° C. After recovery of RNA-containing aqueous phase, one volume of 70% ethanol was added. RNA was obtained using the Qiagen RNeasy kit (Qiagen, Valencia, Calif.) for purification of total RNA from animal cells. RNA (500 ng) was used with the iScript cDNA kit (Bio-Rad) for cDNA synthesis. Quantitative PCR was carried out on a CFX96 real-time PCR detection system (Bio-Rad), using 15 ng equivalent cDNA and SYBR Green qPCR master mix (Bio-Rad). PCR reaction conditions were 3 minutes at 95.0° C., followed by cycles of 10 seconds at 95.0° C., and 30 seconds at 55.0° C. for 39 total cycles (Bio-Rad CFX Manager 3.1 preloaded, CFX-2stepAmp protocol).

Primer sequences used for target amplification were as follows:

(1)
Collagen type III (Col III) (forward)
(SEQ ID NO: 1)
5′-TCTGAAGCTGATGGGATCAA-3′,
(2) Col III (reverse)
(SEQ ID NO: 2)
5′-TCCATTCCCCAGTGTGTTTAG-3′;
(3) collagen type Ia2 (ColIa2) (forward)
(SEQ ID NO: 3)
5′-GCAGGTTCACCTACTCTGTCCT-3′,
(4) CollIa2 (reverse)
(SEQ ID NO: 4)
5′-CTTGCCCCATTCATTTGTCT-3′;
(5) CD41 (forward)
(SEQ ID NO: 5)
5′-AAGCTGAAGCCACAGTGGAG-3′;
(6) CD41 (reverse)
(SEQ ID NO: 6)
5′-TGGAGACCCATCTGTCCAA-3′;
(7) CD61 (forward)
(SEQ ID NO: 7)
5′-GCAAGTACTGTGAGTGCGATG-3′;
(8) CD61 (reverse)
(SEQ ID NO: 8)
5′-CGCAGTCCCCACAGTTACA-3′;
(9) Glyceraldehyde 3-phosphate dehydrogenase
(GAPDH) (forward)
(SEQ ID NO: 9)
5′-CCGGGTTCCTATAAATACGGACTG-3′;
and
(10) GADPH (reverse)
(SEQ ID NO: 10)
5′-GTCTACGGGACGAGGCTGG-3′.

Relative gene expression to the housekeeping genes was calculated using the AACq method as disclosed in Schmittgen, T. D., Analyzing real-time PCR data by the comparative C(T)-method, NAT PROTOC. 2008; 3: 1101-8 and Pfaffl, M. W., A new mathematical model for relative quantification in real-time RT-PCR, NUCLEIC ACIDS RES. 2001; 29: e45.

Statistical Analysis

Statistical analysis was performed using GraphPad Prism 7 (San Diego, Calif.). Results are represented as means±SEM. P values of <0.05 were considered significant. Two-way ANOVA with either Tukey's or Sidak's post-hoc tests were used for multiple comparisons.

### Example 1—Captopril Decreases Reticulin Score and Spleen Weight

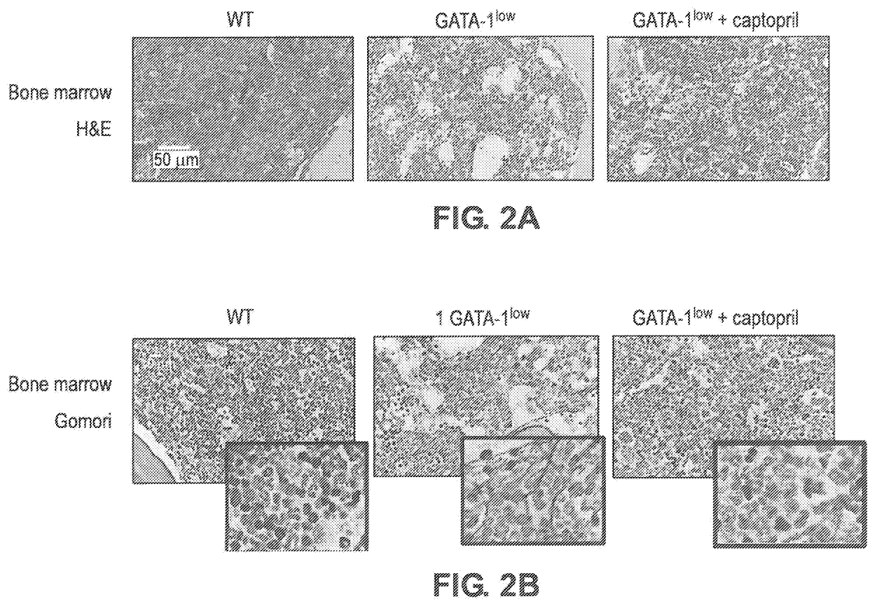

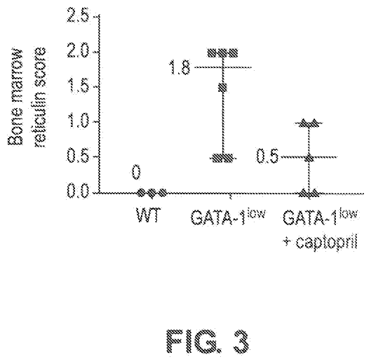

To determine the efficacy of captopril in reversing MF, morphologic and phenotypic changes in the Gata1^{low} mouse model were evaluated. Untreated Gata1^{low} mice at 13 months of age exhibited classic features of marrow MF as compared to wild type CD1 mice that were visually observable in both haematoxylin and eosin staining of bone marrow from the humeri of three mouse subjects, as well as Gomori staining of the same histological sections, which showed reticulin deposition in vehicle-treated Gata1^{low} mice. See FIGS. 2A and 2B. Additional morphologic indications of fibrosis included cellular streaming and dilated sinuses. Megakaryocytes in the bone marrow of the Gata1^{low} mice were abnormally present in patchy clusters and with paratrabecular distribution. The megakaryocytes in the Gata1^{low} mice also displayed moderate megakaryocytic hyperplasia, with atypical morphology and enlarged bulbous nuclei compared with wild type. As shown in FIG. 3, the reticulin score averaged 1.8 out of 3 in the Gata1^{low} mice, in contrast to wild-type mice that scored reticulin as 0 (normal) (p value <0.05 by one-tailed Mann-Whitney test).

Mice treated with captopril for two months, from 10-12 months of age, reduced the severity off bone marrow fibrosis at 13 months of age, with only focal and patchy cellular streaming and rare dilated sinuses. Captopril treated mice had only mild megakaryocytic hyperplasia, with scattered morphologically abnormalities, and displayed only focal megakaryocytic clusters compared with untreated Gata1^{low} mice. See FIGS. 2A and 2B. Treatment with captopril reduced the averaged reticulin score to 0.5 in the Gata1^{low} mice. See FIG. 3.

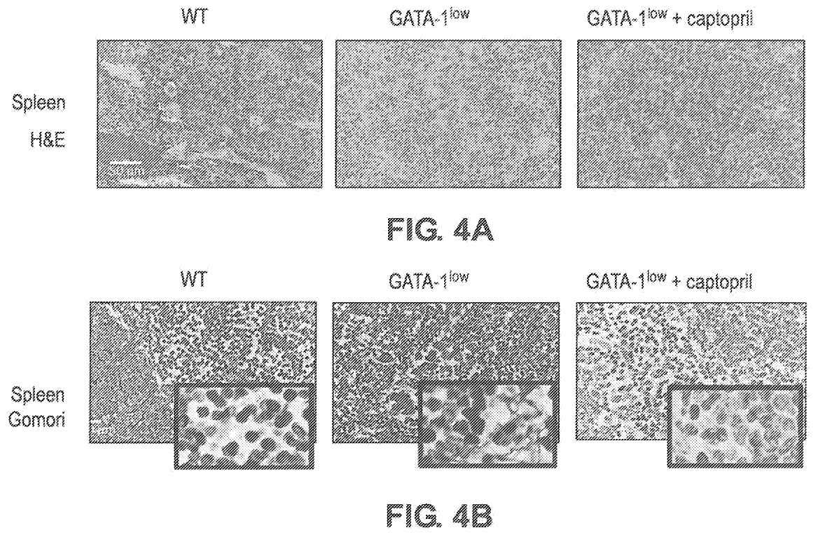

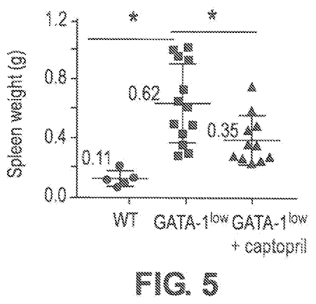

Levels of megakaryocytes and extramedullary hematopoiesis were compared in the spleens of wild-type, untreated Gata1^{low}, and captopril-treated Gata1^{low} mice. Histologically, the untreated Gata1^{low} mice demonstrated significant extramedullary hematopoiesis with increased numbers of enlarged atypical megakaryocytes which were present, in some areas, in large aggregates and sheets. See FIGS. 4A and 4B. As shown in FIGS. 4A and 4B, the captopril-treated Gata1^{low} mice demonstrated moderate amounts of extramedullary hematopoiesis with reduced numbers of atypical megakaryocytes. Consistent with previous reports of splenomegaly in Gata1^{low} mice, the splenic weight was increased 6-fold in untreated Gata1^{low} mice as compared to wt CD1 mice (p value <0.05). See FIG. 5. FIG. 5 further shows that captopril treatment for 2 months induced about a 2-fold decrease (p<0.05) in splenic weight in Gata1^{low} mice as compared to untreated Gata1^{low} mice.

### Example 2—Captopril Stabilized Blood Cells

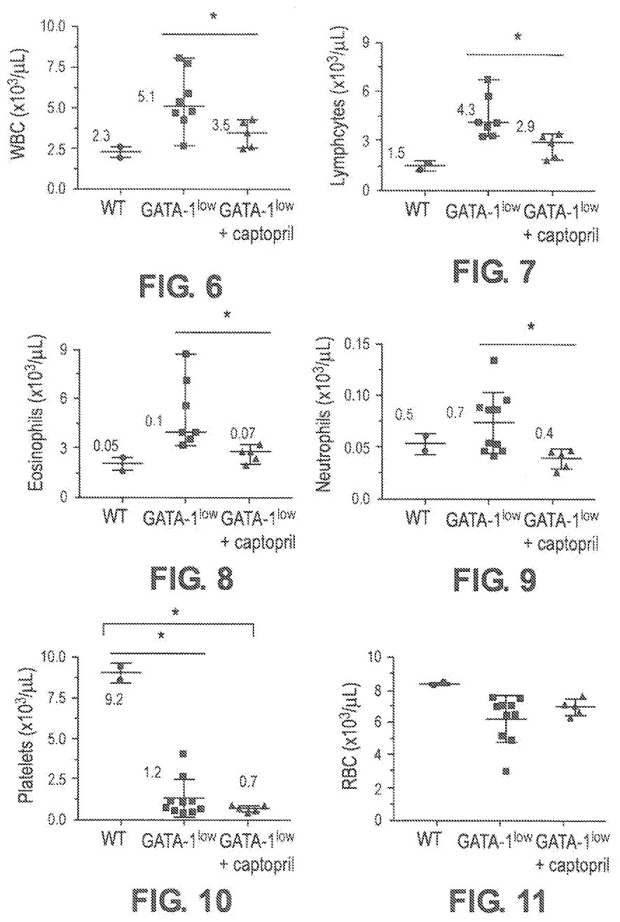

Peripheral blood counts were studied in captopril-treated and untreated Gata1^{low} mice and their wild-type littermates. Wild-type and Gata1^{low} mice were treated from 10 months to 12 months with either 72 mg/kg per day of captopril or vehicle in drinking water. The mice were euthanized at 13.5 months, and tissues were harvested. Complete blood cell counts with differentials were obtained. As shown in FIGS. 6-9, captopril treatment normalized white blood cells (WBC), lymphocytes, eosinophils, and neutrophils compared with untreated Gata1^{low} mice. As shown in FIG. 9, captopril treatment did not ameliorate the platelet count. Likewise, captopril treatment did not ameliorate the mean platelet volume. Gata1^{low} mice have been demonstrated to have reduced platelet numbers, believed to be due to megakaryocyte dysfunction; although captopril reduced the numbers of megakaryocytes, the remaining megakaryocytes were still not functional for platelet production. See FIG. 10. A significant reduction of red blood cells (RBC) in the Gata1^{low} mice was not observed. See FIG. 11. This is consistent with previous findings indicating that the onset of anemia is usually later than 13 months. These data suggest that captopril's effects serve to stabilize the levels of a number of blood cells.

### Example 3—Captopril Reduced Megakaryocytes

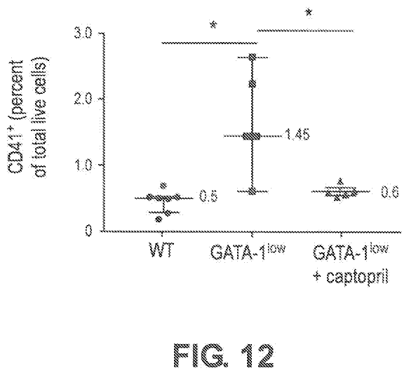

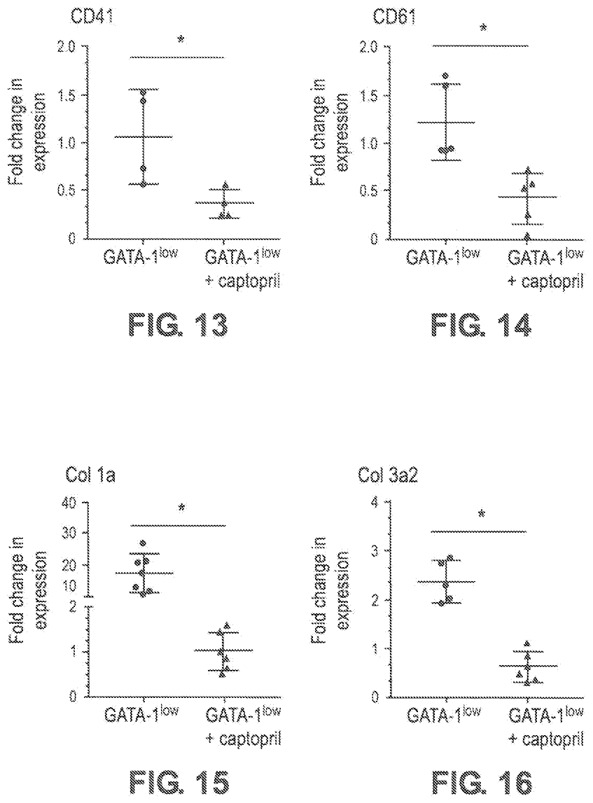

The possible mechanism of action of captopril in the bone marrow and spleen was investigated. Wild-type (wt) or Gata1^{low} mice were treated from 10 to 12 months with either 72 mg/kg/day captopril or vehicle in drinking water. Mice were euthanized at 13 months, and tissues were harvested. Flow cytometric analysis of murine mononuclear cells demonstrated about a 3-fold increase in the frequency of CD115⁻/CD41⁺ megakaryocytes of total live cells in the bone marrow of Gata1^{low} mice compared to wild-type CD1 mice, from 0.5% to 1.45% (p<0.05). Captopril treatment reduced the number of megakaryocytes to 0.6% of total live cells (p<0.05). See FIG. 12. These results were confirmed by qRT-PCR detection of CD41 and CD61 markers, which were decreased approximately 3-fold and 2-fold, respectively, in Gata1^{low} mice treated with captopril as compared to untreated mice (p<0.05). See FIGS. 13-14. There was reduced expression of both Col1a and Col3a2, which decreased approximately 15-fold and approximately 4-fold, respectively (p<0.05). See FIGS. 15-16.

### Example 4—Captopril Reduces Megakaryocytes and Collagen in the Spleen

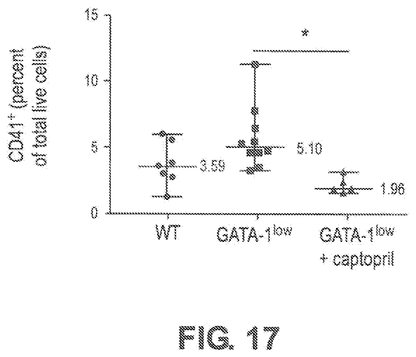

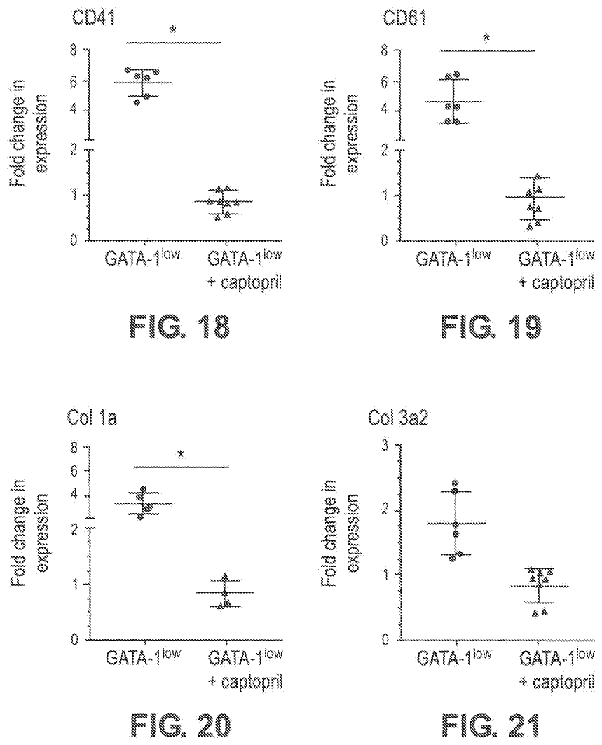

Because of the observed changes in spleen histology and weight from captopril administration, the effect of captopril on megakaryocytes and collagen in the spleens of Gata1^{low} mice was observed. Flow cytometric analysis also showed that Gata1^{low} mice had a trend toward higher levels of splenic megakaryocytes as compared to wild-type CD1 mice (see FIG. 17), although this did not reach significance. Approximately a 2-fold decrease in the frequency of megakaryocytes as a percentage of total live cells in response to captopril treatment (p<0.05) was observed. This decrease in megakaryocytes as determined by fluorescence-activated cell sorting (FACS) was also reflected in qRT-PCR detection of CD41 and CD61 markers, which decreased about 6-fold and about 5-fold, respectively, in captopril treated Gata1^{low} mice (p<0.05). See FIGS. 18-19. Histological observations of the spleen suggested that captopril induced a decrease in collagen fibers, so collagen expression levels in the spleen were investigated. qPCR analysis showed an approximate 4-fold reduction in the level of Col1a expression (p<0.05) and a trend toward reduced Col3a2 expression, although this did not reach significance. See FIGS. 20-21.

### Example 5—Captopril Suppression of EPO, G-CSF and SAA

Animals and ACE Inhibitor Treatment

Mice received total body irradiation (TBI) at a 0.615 Gy/min dose rate in a bilateral gamma radiation field at AFRRI's ⁶⁰Co facility as described in Davis, T. A. et al., Subcutaneous administration of genistein prior to lethal irradiation supports multilineage, hematopoietic progenitor cell recovery and survival, INT. J. RADIAT BIOL. 2007; 83:141-151. Sham irradiated mice were placed in jigs for the same time periods as mice that were irradiated, but did not receive radiation. Captopril, 0.55 g/L or 0.065 g/L (USP grade, Sigma-Aldrich, St. Louis, Mo., USA), was dissolved in acidified water as described above. Captopril consumption was calculated based on volume of water consumed daily and body weights over the time course of the experiment; water intake was reduced in days 0-4 post-irradiation and was maximal at 22-30 days post-irradiation. Captopril at 0.55 g/L in the water was calculated to result in maximal delivery of 58-110/kg/day, and captopril at 0.065 g/L was calculated to result in maximal delivery of 6.8-13 mg/kg/day. Vehicle-treated animals received acidified water with no drug added. Mice were anesthetized with pentobarbital and blood was obtained by cardiocentisis. Complete blood counts (CBC) with differentials were obtained using a Baker Advia 2120 Hematology Analyzer (Siemens, Tarrytown, N.Y., USA). Separate mice were used at each time point (n=5-6).

Statistical Cytokine Levels

Mouse serum samples were obtained by cardiocentesis following euthanasia. Samples were aliquoted and frozen at −80° C. until analysis. Mouse serum was assayed in technical duplicates with a minimum of three biological repeats using either standard ELISAs (R&D Systems, Minneapolis, Minn., USA) or using the electrochemiluminescent MesoScale Discovery (MSD) UPlex (MesoScale Discovery, Gaithersburg, Md., USA). ELISAs were performed according to the manufacturer's instructions with technical duplicates and standard controls for murine granulocyte colony-stimulating factor (G-CSF) and serum amyloid A1 (SAA1). MSD Uplex plates were used to quantitatively measure cytokines, including murine erythropoietin (EPO) and interleukin (IL)-6. All assays were performed according to the manufacturer's instructions with standard controls. The data were acquired on the MSD QuickPlex SQ120 plate reader and analyzed using the Discovery Workbench 3.0 software (MSD). The standard curves for each cytokine were generated using the premixed lyophilized standards provided in the kits. Serial 4-fold dilutions of the standards were run to generate a 7-standard concentration set, and the diluent alone was used as a blank. The cytokine concentrations were determined from the standard curve using a 4-parameter logistic fit curve to transform the mean light intensities into concentrations. The lower limit and upper limit of quantification was determined for each cytokine and all but one sample values fell within the detection ranges of the assays. Those within the detection ranges showed <10% Calc. Conc. CVs.

Statistical Analysis

Kaplan-Meier plots were analyzed using Fisher Exact Tests to assess the differences in survival between the groups after irradiation (GraphPad Prism v7.1, LaJolla, CA, USA). P values lower than 0.05 were considered to be statistically significant. Plasma cytokine levels, hematology results, and gene expression changes were analyzed using two way ANOVA followed by Holm Sidak or Tukey postanalysis (GraphPad Prism v7.1). A value of p <0.05 (two-tailed) was considered statistically significant.

Irradiation and Administration of Captopril

C57BL/6 mice at 12-14 weeks of age were exposed to 7.9 Gy total body ⁶⁰Co irradiation (0.6 Gy/min). Mice received vehicle or captopril (13 mg/kg/day), administered through drinking water either 4 hours post-irradiation for 30 days or 48 hours post-irradiation for 14 days. Blood was obtained at 3, 7, 14, 21, and 30 days post-irradiation for analysis and the following growth factors and cytokines were quantified by MSD or ELISA: EPO, G-CSF, SAA1, and IL-6. Data show means±SEM, n=3-5 per group, except for the 30 day time point for radiation-plus-vehicle, which had only one animal.

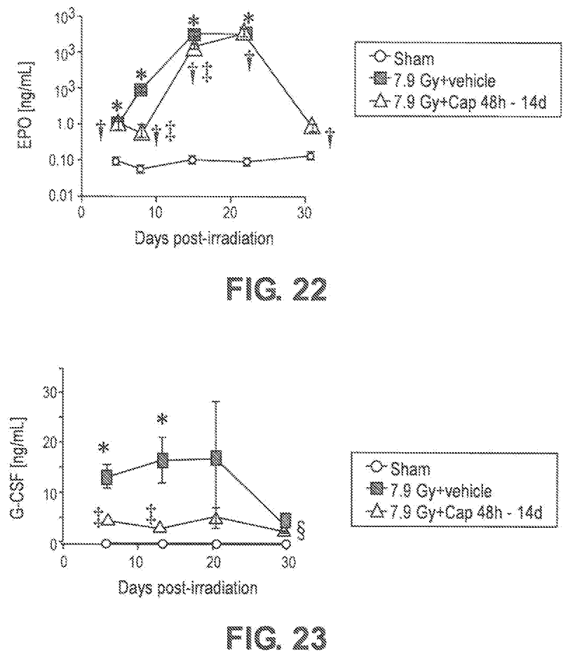

At 4 days post-irradiation, irradiated mice with or without captopril displayed approximately a 10-fold increase in serum EPO. See FIG. 22. At 7 and 14 days post-irradiation, captopril reduced the radiation-induced surge in EPO levels (7 days: radiation-plus-vehicle=8.66±0.9 ng/ml; radiation-plus-captopril=0.57±0.7 ng/ml; 14 days radiation-plus-vehicle=288±140 ng/ml; radiation-plus-captopril=140±24 ng/ml). However, both irradiated groups had similarly elevated EPO levels 21 days post-irradiation. Serum EPO declined sharply in captopril-treated animals by 30 days post-irradiation; no vehicle-treated irradiated animals were alive for comparison at this time point.

Previous studies have demonstrated that other hematopoietic cytokines and growth factors are dramatically increased in response to total body irradiation, including G-CSF, SAA, IL-6, SCF, MIP1a, MIP1b, MCP1, FLT-3, IL-10, IL-1β, IL-2, and KC/CXCL1 [Ossetrova, N. I. et al., Early-response biomarkers for assessment of radiation exposure in a mouse total-body irradiation model, HEALTH PHYS. 2014; 106:772-786]. The observed increases in specific cytokines and growth factors are believed to play a role in natural resistance to radiation damage and may be involved in hematopoietic recovery as well as inflammatory responses after radiation. Accordingly, the effect of 48 hour delayed administration of low-dose captopril on these cytokines and growth factors following 7.9 Gy total body irradiation was investigated.

A significant irradiation-induced increase was observed for G-CSF and SAA that was modulated by delayed captopril treatment. Irradiation caused approximately a 40-fold increase in G-CSF at 7-21 days post-irradiation. Captopril treatment significantly suppressed radiation induced G-CSF at days 7 and 14 post-irradiation (p<0.05). See FIG. 23.

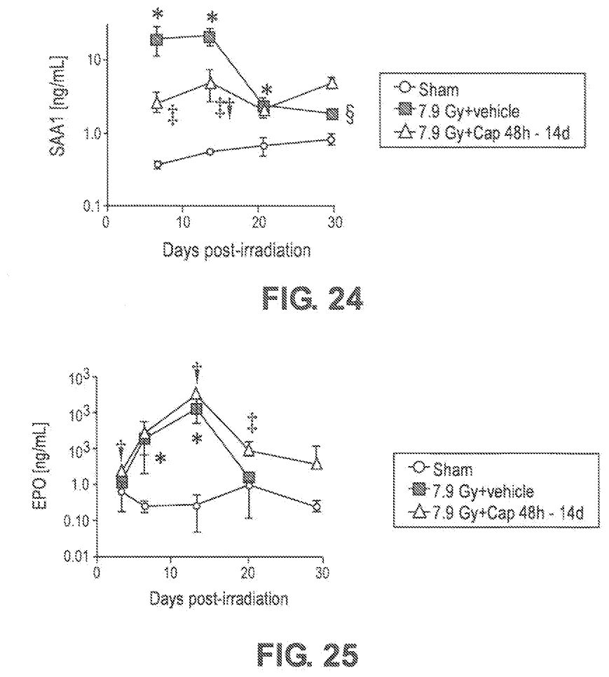

Similarly, irradiation caused approximately a 50-fold increase in SAA1 within 7 days of 7.9 Gy TBI. See FIG. 24. Delayed, low-dose captopril treatment significantly attenuated radiation-induced SAA1 levels on days 7 and 14 post-irradiation (p<0.05). FIG. 24. Note that at 30 days post-irradiation, only one animal remained in the irradiated vehicle-treated group, and statistical significance could not be determined.

IL-1β and IL-6 are known to be primary regulators of SAA1 following acute injury. A significant elevation of IL-1β over the time course examined was not detected (data not shown), so the effect of captopril on IL-6 post-irradiation was investigated. Radiation significantly increased IL-6 levels on days 7 and 14 post-irradiation, but captopril treatment did not significantly suppress IL-6 at any time points. FIG. 25. These data suggest that captopril does not suppress radiation-induced SAA1 through the regulation of either IL-1β or IL-6.

1. A method of treating a myeloproliferative neoplasm in a subject in need thereof, the method comprising administering to the subject a compound chosen from angiotensin converting enzyme (ACE) inhibitors, angiotensin receptor blockers (ARBs), and renin inhibitors, wherein the compound is administered in an amount effective to treat the myeloproliferative neoplasm in the subject.

2. A method of stabilizing white blood cell numbers and/or stabilizing the levels of Interleukin-9 (IL-9) and Stem Cell Factor (SCF) in a patient having a myeloproliferative neoplasm, the method comprising administering to the patient a compound chosen from angiotensin converting enzyme (ACE) inhibitors, angiotensin receptor blockers (ARBs), and renin inhibitors, wherein the compound is administered in an amount effective to stabilize white blood cell numbers and/or stabilize the levels of IL-9 and/or SCF in the patient.

3. The method of claim 2, wherein the white blood cells are one or more of eosinophils, neutrophils, or lymphocytes.

4. A method of stabilizing the levels of at least one hematopoietic growth factor and/or at least one serum amyloid A (SAA) protein in a patient having a myeloproliferative neoplasm, the method comprising administering to the patient a compound chosen from ACE inhibitors, ARBs, and renin inhibitors, wherein the compound is administered in an amount effective to stabilize the levels of at least one hematopoietic growth factor and/or stabilize the levels of at least one SAA protein in the patient.

5. The method of claim 4, wherein the at least one hematopoietic growth factor is selected from the group consisting of erythropoietin (EPO) and granulocyte colony-stimulating factor (G-CSF).

6. The method of claim 4 or 5, wherein the at least one SAA protein is SAA1.

7. A method of stabilizing megakaryocytes in at least one of bone marrow and spleen in a patient having a myeloproliferative neoplasm, the method comprising administering to the patient a compound chosen from ACE inhibitors, ARBs, and renin inhibitors, wherein the compound is administered in an amount effective to stabilize megakaryocytes in at least one of bone marrow and spleen of the patient.

8. The method of claim 1, wherein the myeloproliferative neoplasm is chosen from chronic myeloid leukemia, polycythemia vera, essential thrombocytosis, myelofibrosis, chronic neutrophilic leukemia, chronic eosinophilic leukemia, and hypereosinophilic syndrome.

9. The method of claim 1, wherein the myeloproliferative neoplasm is myelofibrosis and wherein the myelofibrosis is primary or secondary myelofibrosis.

10. The method of claim 9, wherein the myelofibrosis is primary myelofibrosis.

11. The method of claim 1, wherein the compound is an ACE inhibitor.

12. The method of claim 11, wherein the ACE inhibitor is captopril.

13. The method of claim 1, wherein the subject is a mammal.

14. The method of claim 13, wherein the mammal is a human.

15. The method according to claim 1, wherein the administration of the compound stabilizes expression of CD41 and/or CD61 proteins in at least one of bone marrow and spleen of the subject.

16. The method according to claim 1, wherein the administration of the compound stabilizes expression of Col1a and/or Col3a2 in at least one of bone marrow and spleen of the subject.

17. The method according to claim 1, wherein the administration of the compound stabilizes reticulin and/or collagen production in at least one of bone marrow and spleen of the subject.

18. The method according to claim 1, wherein the compound is administered in an amount effective to stabilize splenomegaly in the subject.

19. The method according to claim 1, wherein the compound is administered in an amount effective to stabilize bone marrow fibrosis in the subject.


SEQUENCE LISTING (10 sequences)

SEQ ID NO: 1 - DNA, 20 bp, Artificial Sequence
  Description of Artificial Sequence Synthetic primer
tctgaagctgatgggatcaa

SEQ ID NO: 2 - DNA, 21 bp, Artificial Sequence
  Description of Artificial Sequence Synthetic primer
tccattccccagtgtgtttag

SEQ ID NO: 3 - DNA, 22 bp, Artificial Sequence
  Description of Artificial Sequence Synthetic primer
gcaggttcacctactctgtcct

SEQ ID NO: 4 - DNA, 20 bp, Artificial Sequence
  Description of Artificial Sequence Synthetic primer
cttgccccattcatttgtct

SEQ ID NO: 5 - DNA, 20 bp, Artificial Sequence
  Description of Artificial Sequence Synthetic primer
aagctgaagccacagtggag

SEQ ID NO: 6 - DNA, 19 bp, Artificial Sequence
  Description of Artificial Sequence Synthetic primer
tggagacccatctgtccaa

SEQ ID NO: 7 - DNA, 21 bp, Artificial Sequence
  Description of Artificial Sequence Synthetic primer
gcaagtactgtgagtgcgatg

SEQ ID NO: 8 - DNA, 19 bp, Artificial Sequence
  Description of Artificial Sequence Synthetic primer
cgcagtccccacagttaca

SEQ ID NO: 9 - DNA, 24 bp, Artificial Sequence
  Description of Artificial Sequence Synthetic primer
ccgggttcctataaatacggactg

SEQ ID NO: 10 - DNA, 19 bp, Artificial Sequence
  Description of Artificial Sequence Synthetic primer
gtctacgggacgaggctgg